(19) United States
(12) Patent Application Publication
(10) Pub. No.: US 2006/0141078 A1
(43) Pub. Date: Jun. 29, 2006

(54) Composition comprising a rice protein hydrolysate and an agent for increasing glycosaminoglycan synthesis

(75) Inventors: Veronique Guillou, Antony (FR); Annabelle Thiry, Paris (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 11/266,287

(22) Filed: Nov. 4, 2005

Related U.S. Application Data
(60) Provisional application No. 60/643,172, filed on Jan. 13, 2005.

(30) Foreign Application Priority Data
Nov. 10, 2004 (FR) 04 52593

(51) Int. Cl. A61K 36/899

(52) U.S. Cl. 424/750

(57) ABSTRACT

The present invention relates to a process for preventing and/or reducing the signs of ageing of the skin, in particular for remodelling the face and/or limiting the age-related hollowing of the face, by applying to facial skin a composition containing, in a physiologically acceptable medium, at least one rice protein hydrolysate and at least one agent for increasing glycosaminoglycan synthesis.

### REFERENCE TO PRIOR APPLICATIONS

This application claims priority to U.S. provisional application 60/643,172 filed Jan. 13, 2005, and to French patent application 0452593 filed Nov. 10, 2004, both incorporated herein by reference.

### FIELD OF THE INVENTION

The present invention relates to a method for preventing or delaying the appearance of the signs of ageing of the skin and/or reducing the signs of ageing of the skin, in particular for remodelling the face and/or limiting the age-related hollowing of the face, by applying to facial skin a composition comprising, in a physiologically acceptable medium, at least one rice protein hydrolysate and at least one agent for increasing glycosaminoglycan synthesis.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

The invention also relates to a cosmetic composition comprising, in a physiologically acceptable medium, at least one rice protein hydrolysate and at least one agent for increasing glycosaminoglycan synthesis, chosen from an extract of Laminaria ochroleuca and C-β-D-xylopyranoside-2-hydroxypropane, and the salts and optical and geometrical isomers thereof.

The invention also relates to the cosmetic use of at least one rice protein hydrolysate combined with at least one agent for increasing glycosaminoglycan synthesis, in a composition, as an agent for remodelling the face and/or for limiting the age-related hollowing of the face.

### BACKGROUND OF THE INVENTION

It is known that the signs of ageing of the skin have both chronological causes (genetically programmed ageing) and additional aggravating factors such as exposure to UV radiation and, above all, hormone deficiencies occurring during the menopause. They are reflected by changes in the state of the skin that are essentially due to a slowing-down and disequilibrium of cutaneous functioning, which is reflected by “atrophy” of all of the strata of the skin. Thus, a decrease in the quality of the dermis (elastin, collagen and glycosaminoglycans) and loss of consistency of the extracellular matrix; a decrease in the thickness of the epidermis (slowing-down of cell renewal); slowing-down of the production of epidermal structure proteins and lipids; disequilibrium of desquamation; and a reduction in the water content of the skin.

In particular, following the above-mentioned modifications, the skin's supporting mattress becomes weakened and the adipose mass melts: thus, the face gradually becomes hollowed out and the skin has a tendency to sag. This phenomenon is especially visible on the cheeks, which become hollowed more and more, the area around the eyes, and the oval of the face, which becomes less well defined (appearance of jowls).

Now, means are constantly being sought to combat, or at the very least delay, the onset of these signs of ageing and thus to look younger for longer.

To limit this sagging of the skin and the appearance of the hollows mentioned above, one well known means consists in reinforcing the architecture of the dermis by means of active agents that stimulate the synthesis of collagen and of glycosaminoglycans (referred to hereinbelow as GAG). Active agents conventionally used for this purpose are especially: ascorbic acid and its derivatives, Centella asiatica extracts, retinoids, certain soybean protein hydrolysates and auxins (which increase collagen synthesis), and also extracts of brown alga Padina pavonica and Saccharomyces cerevisiae (which increase GAG synthesis). However, the result obtained via this route is not entirely satisfactory.

### SUMMARY OF THE INVENTION

The inventors have now discovered, entirely surprisingly and unexpectedly, that rice protein hydrolysates comprising peptides of low molecular weight (less than 1400 daltons), such as those described in WO 02/102 347 and/or peptides with a molecular weight of greater than 1400 daltons, have the property of inhibiting lipolysis and thus of preventing or slowing down the shedding of the fats contained in the support tissues of the skin.

It is in this context that the inventors discovered using a rice protein hydrolysate in combination with an agent for increasing glycosaminoglycan synthesis in order to effectively combat the signs of ageing of the skin and in particular to combat the various mechanisms of hollowing out and sagging of the face. Of course, it is realized that chronological ageing is impossible to stop, and such implication is not intended herein. Rather, the invention relates to a method and composition for preventing or delaying the appearance of the signs of ageing of the skin and/or reducing existing signs of ageing of the skin, in particular for remodelling the face and/or limiting the age-related hollowing of the face, as well as to other uses described herein.

The prior art discloses the use of rice protein hydrolysates in cosmetic compositions for the hair or the skin, especially as a moisturizer (EP 1 090 630) or as a anti-wrinkle agent (WO 02/102 347).

However, to the inventors' knowledge, it has never been suggested to use a rice hydrolysate in combination with an agent for increasing glycosaminoglycan synthesis to combat the signs of ageing of the skin, and in particular to remodel the face and/or to limit the age-related hollowing of the face.

One subject of the present invention is thus a process for preventing and/or reducing the signs of ageing of the skin, by applying to facial skin a composition comprising, in a physiologically acceptable medium, at least one rice protein hydrolysate and at least one agent for increasing glycosaminoglycan synthesis.

In particular, the process according to the invention is directed towards remodelling the face and/or limiting the age-related hollowing of the face.

More specifically, the cosmetic process according to the invention is directed towards redefining the contours of the face and/or limiting the hollowing of the cheeks and/or of the contour of the eyes.

In this regard, the invention method and composition is preferably used by subjects desirous of the benefits noted herein, subjects “in need of” these benefits. Such subjects are typically suffering from signs of ageing of the skin generally, including from age-related hollowing of the face and/or cheeks, or from age-related changes to the contour of the eyes, such as by self diagnosis or cosmetician or medical diagnosis, or are at a recognized and appreciated risk of developing such conditions and who use the invention methods and compositions to combat these effects. In this regard, the invention process can be viewed as one for delaying the onset of the appearance of, and/or for reducing signs of, ageing of the skin.

Naturally, one using the invention as disclosed will use an amount of the invention composition effective to reduce the signs of ageing. Such amount is inclusive of an amount of the compositions described herein at the disclosed concentrations of active ingredients sufficient to cover the area of the skin being treated in a single application, and of course includes that amount applied upon repeated application, for example on a daily basis over a course of days, weeks, etc. In a preferred embodiment the invention process includes multiple applications of the invention composition to the area(s) of skin in need of attention.

Rice Protein Hydrolysate

The rice protein hydrolysate used in the process of the invention may comprise peptides of low molecular weight (less than 1400 daltons) and/or peptides with a molecular weight of greater than 1400 daltons.

In particular, a rice protein hydrolysate comprising peptides with a molecular mass (weight) of less than or equal to 5000 daltons, or preferentially a molecular mass of less than or equal to 3000 daltons, will be used.

A process for preparing this rice protein hydrolysate comprises, for example:

grinding rice grains (e.g.: Oryza sativa),

dissolving the rice proteins in water to obtain a suspension,

hydrolysing this suspension in the presence of one or more proteases,

advantageously, filtering through a membrane to remove the insoluble compounds, the residual enzyme and the high molecular weight peptides (greater than 10 000 daltons), and

optionally, performing a sterilizing filtration on this concentrate.

According to one particular mode of the invention, the rice protein hydrolysate used in the process of the invention contains peptides with an average molecular mass ranging from 1000 daltons to 5000 daltons and preferably an average molecular mass ranging from 2000 daltons to 3000 daltons.

A process for preparing this rice protein hydrolysate comprises, for example:

grinding rice grains (e.g.: Oryza sativa),

dissolving the rice proteins in water to obtain a suspension,

hydrolysing this suspension in the presence of one or more proteases,

filtering through a membrane to remove the insoluble compounds, the residual enzyme and the high molecular weight peptides, so as to concentrate the peptides with a molecular mass ranging from 1000 to 5000 daltons and preferably from 2000 to 3000 daltons in the active fraction, and

optionally, performing a sterilizing filtration on this concentrate.

Advantageously, the process for preparing this rice protein hydrolysate does not comprise a fermentation or germination step. In addition, the rice used in the composition according to the invention and/or used in the above process preferably belongs to the genus Oryza and is more preferentially the species Oryza sativa L. Advantageously, the rice hydrolysate used in the composition according to the invention, and that obtained in the above preparation process, are derived from a rice fraction free of rice bran.

This rice protein hydrolysate may especially be defined by the following amino acid profile (% relative to the total % of amino acids):

| | | | |
|---|---|---|---|
| Ile | 4.5 | Met | 2.3 |
| Leu | 8.4 | Lys | 4.5 |
| Val | 6.3 | Arg | 9.0 |
| Met | 2.3 | Phe | 5.8 |
| Ala | 5.6 | Tyr | 5.3 |
| Gly | 4.3 | Glu | 17.5 |
| Ser | 5.5 | Asp | 10.0 |
| Thr | 3.9 | Cys | 0.9 |
| Pro | 3.9 | | |

Such a rice protein hydrolysate is referenced in Chemical Abstracts under the CAS No. 94350-05-7; it is especially commercially available from the company Pentapharm under the trade name Colhibin®.

According to another embodiment of the invention, the rice protein hydrolysate used in the process of the invention is enriched in peptides with a molecular mass of less than 1400 daltons.

Preferably, more than 50% and better still more than 70% of the peptides contained in the rice protein hydrolysate have a mass of less than 1400 daltons. In addition, the peptides contained in the rice protein hydrolysate preferably have a mean molecular mass of between 300 and 400 daltons and advantageously between 350 and 370 daltons. Finally, it is preferable for at least 40% of the peptides having a molecular mass of less than 1400 daltons to have a molecular mass of between 75 and 175 daltons (“short peptides”). In general, less than 60%, or even less than 50%, of the peptides having a molecular mass of less of 1400 daltons will have a molecular mass of between 75 and 175 daltons.

In other words, it is preferable for at least 20%, and more preferentially at least 30%, of the peptides contained in this rice protein hydrolysate to have a molecular mass of between 75 and 175 daltons. These low molecular mass peptides will generally represent less than 50%, or even less than 40%, of the peptides contained in the rice protein hydrolysate. They preferably consist essentially of mono-, di- and tripeptides.

Such a hydrolysate is especially commercially available from the company Silab under the trade name Nutriskin. It may be obtained as described in patent application WO 02/102 347.

A process for preparing this rice protein hydrolysate comprises, for example:

dissolving rice proteins in water to obtain a suspension,

hydrolysing this suspension in the presence of one or more enzymes chosen from: bacterial aminopeptidases in neutral or acidic medium, stomach proteases of animal origin in acidic medium, bacterial proteases in neutral medium, bacterial proteases in alkaline medium, extracted from Bacillus licheniformis or Bacillus subtilis, bacterial endoproteases, and pancreatic enzymes of animal origin;

concentrating the peptides with a molecular weight of less than 1400 daltons in the active fraction; and

optionally, performing a sterilizing filtration on this concentrate.

Advantageously, this process does not comprise a fermentation or germination step. In addition, the rice used in the composition according to the invention and/or used in the above process preferably belongs to the genus Oryza and is more preferentially the species Oriza sativa L. Advantageously, the rice hydrolysate used in the composition according to the invention, and that obtained in the above process, are derived from a rice fraction free of rice bran.

The rice peptide extracts are generally used in the present invention in an amount that is effective to inhibit the in vitro hydrolysis of adipocyte triglycerides, according to a test similar to that presented in Example 1 below.

It may thus represent, for example, from 0.01% to 2% by weight (of active material) and better still from 0.02% to 0.4% by weight (of active material) relative to the total weight of the composition. Preferably, it represents from 0.01% to 0.5% by weight (of active material) and better still from 0.02% to 0.2% by weight (of active material) relative to the total weight of the composition.

Agent for Increasing Glycosaminoglycan Synthesis

Agents for increasing glycosaminoglycan synthesis that may especially be used according to the invention include: D-xylose, its esters and oligosaccharides containing D-xylose as described especially in patent application WO 99/24009; the C-glycosides described in patent application WO 02/051 828, such as C-β-D-xylopyranoside-2-hydroxypropane and the salts and optical and geometrical isomers thereof; a product of fermentation of milk with Lactobacillus vulgaris, such as the product sold by the company Brooks under the trade name Biomin yogourth®; an extract of brown alga Padina pavonica, such as the product sold by the company Alban Müller under the trade name HSP3®; an extract of Saccharomyces cerevisiae available especially from the company Silab under the trade name Firmalift® or from the company LSN under the trade name Cytovitin®; an extract of Laminaria ochroleuca, such as the product available from the company Secma under the trade name Laminaine®; an extract of Centella asiatica such as the product available from the company Roche under the trade name Etca®; and an extract of cress (Nasturtium officinale) available from the company Silab under the trade name Odraline®.

Preferably, at least one agent for increasing glycosaminoglycan synthesis chosen from D-xylose, its esters and oligosaccharides containing D-xylose, the C-glycosides described in patent application WO 02/051 828, such as C—-D-xylopyranoside-2-hydroxypropane and the salts and optical and geometrical isomers thereof, an extract of brown alga Padina pavonica, an extract of Laminaria ochroleuca and extract of cress (Nasturtium officinale) are used.

In the context of this invention, it is preferred to use as agent for increasing glycosaminoglycan synthesis an extract of Laminaria ochroleuca or C-β-D-xylopyranoside-2-hydroxypropane, and the salts and optical and geometrical isomers thereof.

Laminaria ochroleuca is a brown-yellow coloured variety of laminaria, which is known to combat oxidative stress and cell ageing and in particular to promote the synthesis of glycosaminoglycans (EP-1 074 262).

The extract of Laminaria ochroleuca is especially obtainable according to a process as described in patent application EP-1 074 262, and which comprises, for example:

harvesting fresh algae, for example from the northern coastline of Brittany,

washing them,

milling them, advantageously to a size of between 100 and 200 μm,

removing the cell debris by centrifugation, to obtain a first supernatant,

fractionating the supernatant by precipitation with acid, preferably with hydrochloric acid, and by adsorption onto activated charcoal, to obtain a second supernatant,

filtering the second supernatant (for example by means of tangential filtration with a cut-off threshold of 1000 daltons) until the filtrate has reached a glycine-betaine concentration, measured by HPLC, of 12.5%±2.5%.

C-glycosides such as C-β-D-xylopyranoside-2-hydroxypropane, and the salts and optical and geometrical isomers thereof, are described in patent application WO 02/051 828, which is incorporated by reference into the present patent application.

The agent for increasing glycosaminoglycan synthesis may represent, for example, from 0.001% to 5% by weight (of active material) and preferably from 0.01% to 1% by weight (of active material) relative to the total weight of the composition. When it is an extract of Laminaria ochroleuca, this agent advantageously represents from 0.005% to 0.1% (by weight of solids) and preferably from 0.01% to 0.05% (by weight of solids) relative to the total weight of the composition.

In the text hereinabove, the term “active material” means a compound or a mixture of compounds lacking solvent in which, and lacking additives (such as preserving agents) with which, they are sold and lacking solvent in which they have been synthesized or by means of which they have been extracted.

The invention also relates to a composition that may be used in the process according to the invention, comprising, in a physiologically acceptable medium, at least one rice protein hydrolysate and at least one extract of Laminaria ochroleuca.

The invention also relates to a cosmetic composition that may be used in the process according to the invention, comprising, in a physiologically acceptable medium, at least one rice protein hydrolysate and at least C-β-D-xylopyranoside-2-hydroxypropane, and the salts and optical and geometrical isomers thereof.

The rice protein hydrolysate and the extract of Laminaria ochroleuca and the C-glycosides that may be used in the said composition are defined hereinabove in the description.

The rice protein hydrolysate represents in these compositions, for example, from 0.01% to 2% by weight (of active material) and better still from 0.02% to 0.4% by weight (of active material) relative to the total weight of the composition.

Preferably, it represents from 0.01% to 0.5% by weight (of active material) and better still from 0.02% to 0.2% by weight (of active material) relative to the total weight of the composition. The extract of Laminaria ochroleuca represents from 0.001% to 5% by weight (of active material) and preferably from 0.1% to 1% by weight (of active material) relative to the total weight of the composition. When it is an extract of Laminaria ochroleuca, this agent advantageously represents from 0.005% to 0.1% (by weight of solids) and preferably from 0.01% to 0.05% (by weight of solids) relative to the total weight of the composition.

The C-glycosides and in particular C-β-xylopyranoside-2-hydroxypropane, and the salts and optical and geometrical isomers thereof, represent, for example, from 0.001% to 5% by weight (of active material) relative to the total weight of the composition and preferably from 0.01% to 1% by weight (of active material).

The composition according to the invention is preferably suitable for topical application to the skin and thus generally comprises a physiologically acceptable medium, i.e. a medium that is compatible with the skin and/or its integuments. It is preferably a cosmetically acceptable medium, i.e. a medium that has an acceptable colour, odour and feel and does not cause any unacceptable discomfort (stinging, tautness or redness) liable to put the user off using this composition.

The composition according to the invention may be in any form, including any galenical form such as those used for topical application, and especially in the form of dispersions of the lotion or aqueous gel type, emulsions of liquid or semi-liquid consistency of the milk type, obtained by dispersing a fatty phase in an aqueous phase (O/W) or, conversely, (W/o), or suspensions or emulsions of soft, semi-solid or solid consistency of the cream or gel type, or alternatively multiple emulsions (W/O/W or O/W/O), microemulsions, vesicular dispersions of ionic and/or nonionic type, or wax/aqueous phase dispersions. These compositions are prepared according to the usual methods.

According to one preferred embodiment of the invention, the composition is in the form of an oil-in-water (O/W) emulsion. In addition, according to one variant of the invention, the rice protein hydrolysate and the agent for increasing glycosaminoglycan synthesis are incorporated together, or each separately, into a vesicle of liposome type or of niosome type, in particular in niosomes as described especially in patent application EP-0 582 503. These niosomes are formed from a polyol ether or ester, such as a fatty acid ester of sucrose, in particular sucrose distearate; from an anionic or cationic amphiphilic lipid, in particular an anionic amphiphilic lipid, such as a stearoyl glutamate salt (especially the disodium salt); and from a sterol such as cholesterol.

As oils that may be used in the composition according to the invention, mention may be made of: esters of fatty acids and of fatty alcohols; volatile silicone oils (such as cyclomethicones) or non-volatile silicone oils (such as dimethicones); branched fatty alcohols such as octyldodecanol; hydrocarbon-based oils such as petroleum jelly, squalane, isohexadecane and mineral oils; plant oils; and shea butter. The oily phase may also comprise waxes such as beeswax.

This composition may also contain various adjuvants commonly used in cosmetics, such as polyols, in particular glycerol, propylene glycol and polyethylene glycols; emulsifiers including fatty acid esters of polyethylene glycol, fatty acid esters of glycerol, fatty acid esters of sucrose, fatty alcohol ethers of polyethylene glycol, and fatty acid esters of methylglucose, which are optionally oxyethylenated; coemulsifiers such as cetyl alcohol and stearyl alcohol; fillers, especially silica, and expanded powders such as microspheres formed from a terpolymer of vinylidene chloride, acrylonitrile and methacrylate, sold under the name Expancel by the company Kemanord Plast; thickeners and/or gelling agents such as polysaccharide or silicone gums, acrylamide homopolymers and copolymers, acrylic acid homopolymers and copolymers, and acrylamidomethylpropanesulfonic acid (AMPS) homopolymers and copolymers; preserving agents; sequesterants; pH regulators such as triethanolamine, sodium hydroxide or citric acid; ethanol; dyes; nacres; and fragrances.

Needless to say, a person skilled in the art will take care to select this or these optional additional compound(s) and/or the amount thereof such that the advantageous properties of the composition according to the invention are not, or are not substantially, adversely affected by the envisaged addition.

The composition according to the invention makes it possible to slow down the loss of material from the face that is responsible for its hollowing and its sagging with age. To reinforce the effects of this composition, it may contain at least one compound chosen from:

tensioning agents such as:

(1) polyurethane latices or acrylic-silicone latices, in particular those described in patent application EP-1 038 519, such as a polydimethylsiloxane grafted propylthio(polymethyl acrylate), propylthio(polymethyl methacrylate) and propylthio(polymethacrylic acid), or alternatively a polydimethylsiloxane grafted propylthio(polyisobutyl methacrylate) and propylthio(polymethacrylic acid). Such grafted silicone polymers are especially sold by the company 3M under the trade names VS 80, VS 70 and L021,

(2) polymers of natural origin, especially (a) polyholosides, for example (i) in the starch form derived especially from rice, corn, potato, cassaya, pea, Triticum aestivum wheat, oat, etc. or (ii) in the form of carrageenans, alginates, agars, gellans, cellulose-based polymers and pectins, advantageously as an aqueous dispersion of gel microparticles, and (b) latices consisting of shellac resin, sandarac gum, dammar resins, elemi gums, copal resins and cellulose derivatives, and mixtures thereof,

(3) plant proteins and plant protein hydrolysates, in particular from corn, rye, Triticum aestivum wheat, buckwheat, sesame, spelt, pea, bean, lentil, soybean and lupin,

(3) mixed silicates, especially phyllosilicates and in particular laponites,

(4) wax microparticles chosen, for example, from carnauba wax, candelilla wax and esparto grass wax,

(5) colloidal particles of mineral filler with a number-average diameter of between 0.1 and 100 nm and preferably between 3 and 30 nm, and chosen, for example, from: silica, silica-alumina composites, cerium oxide, zirconium oxide, alumina, calcium carbonate, barium sulfate, calcium sulfate, zinc oxide and titanium dioxide;

dermo-relaxing agents such as adenosine, magnesium and manganese salts (in particular gluconates) and extracts of Dioscorea opposita or Dioscorea villosa containing diosgenin;

agents for increasing the synthesis of proteins of the dermo-epidermal junction, such as laminin, collagen IV and/or collagen VII, and in particular ascorbic acid or a malt hydrolysate, obtained by enzymatic hydrolysis, such as the product sold under the trade name Basaline by the company Coletica;

moisturizers, which may be either humectants such as hyaluronic acid and its salts, or agents for stimulating keratinocyte differentiation and thus for reinforcing the skin barrier to prevent drying-out of the skin, such as: a peptide extract of lupin such as the product sold by the company Silab under the trade name Structurine®; sodium β-sitosteryl sulfate, such as the product sold by the company Seporga under the trade name Phytocohesine®; a water-soluble extract of corn, such as the product sold by the company Solabia under the trade name Phytovityl®; a peptide extract of Voandzeia substerranea, such as the product sold by the company Laboratoires Serobiologiques under the trade name Filladyn LS 9397®; and lignans such as secoisolariciresinol;

desquamating agents such as α- and β-hydroxy acids, in particular salicylic acid and its derivatives such as 5-n-octanoylsalicylic acid; and an extract of Fagus sylvatica beech buds, such as the product sold by the company Gattefosse under the trade name Gatuline®; and

compounds for increasing collagen and procollagen synthesis, such as monomethylsilanetriol mannuronate, sold especially by the company Exsymol under the trade name Algisium C.

The composition according to the invention may also contain at least one UVA and/or UVB screening agent. The sunscreens may be chosen from organic screening agents and mineral screening agents, and mixtures thereof.

The organic screening agents that are more particularly preferred are chosen from the following (cited according to the CTFA nomenclature): ethylhexyl salicylate, homosalate, ethylhexyl methoxycinnamate, butylmethoxydibenzoylmethane, octocrylene, phenylbenzimidazolesulfonic acid, disodium phenyldibenzimidazoletetrasulfonate, benzophenone-3, benzophenone-4, benzophenone-5,4-methylbenzylidene-camphor, terephthalylidenedicamphorsulfonic acid, bis-ethylhexyloxyphenol methoxyphenyl triazine, ethylhexyl triazone, diethylhexylbutamidotriazone, methylene bis(benzotriazolyl)tetramethylbutylphenol, drometrizole trisiloxane, polysilicone-15, and from the following (cited as chemical names): n-hexyl 2-(4-diethylamino-2-hydroxybenzoyl)benzoate, 1,1-dicarboxy(2,2′-dimethylpropyl)-4,4-diphenylbutadiene, 2,4-bis[5-1(dimethylpropyl)benzoxazol-2-yl(4-phenyl)imino]-6-(2-ethylhexyl)imino-1,3,5-triazine, and mixtures thereof.

The mineral screening agents are preferably chosen from pigments or nanopigments (mean size of the primary particles: generally between 5 nm and 100 nm and preferably between 10 nm and 50 nm) of coated or uncoated metal oxides, for instance titanium oxide (amorphous or crystallized in rutile and/or anatase form), iron oxide, zinc oxide, zirconium oxide or cerium oxide nanopigments.

The invention also relates to the cosmetic use of at least one rice protein hydrolysate combined with at least one agent for increasing glycosaminoglycan synthesis, in a composition, as an agent for remodelling the face and/or for limiting the age-related hollowing of the face.

The invention will now be illustrated by means of the non-limiting examples that follow. In these examples, the amounts are indicated as weight percentages.

### EXAMPLES

### Example 1

### Demonstration of the Properties of a Rice Protein Hydrolysate on Lipolysis

We evaluated the effect of a rice protein hydrolysate according to the invention (Nutriskin from Silab, defined in patent application WO 02/102 347, at 6.25% active material) on the hydrolysis of adipocyte triglycerides by measuring the release of non-esterified fatty acids (NEFA) into the medium.

This rice protein hydrolysate comprises peptides of low molecular weight (less than 1400 daltons).

Two references were used: theophylline (which stimulates the production of cyclic AMP) and isoproterenol (which is a β-adrenergic agonist). These two molecules have a lipolytic effect, i.e. they increase the release of non-esterified fatty acids by the adipocytes.

Procedure:

The adipocytes were isolated from an abdominal plasty and then incubated in the presence of collagenase, washed and taken up in test medium. This test medium comprised:
test product (hydrolysate representing 0.0625% or 0.0125% of active material/theophylline 1 mM/isoproterenol 1 μM), or nothing for the control
bicarbonate 1.87 mg/ml
penicillin/streptomycin 25 IU/ml/25 μg/ml
glutamine 2 mM
MEM without phenol red qs 100% (v/v) supplemented with 0.5% (w/v) defatted bovine serum albumin

We then added the adipocyte suspension and the mixtures were incubated at 37° C. The NEFAs were assayed in the fractions of sub-adipocyte medium, after decantation. The assay was performed using an NEFA-C kit according to the manufacturer's instructions.

Two controls (a positive and a negative) of absence of interference of the product with the NEFA assay were performed.

Results:

| | | NEFA | % activity | |
|---|---|---|---|---|
| Treatment | Concentration | (μM) | vs Control | p |
| Untreated | — | 78.8 | 100 | P < 0.01 |
| control | | | | |
| Rice protein | 0.0625% | 32.0 | 41 | P < 0.01 |
| hydrolysate | 0.0125% | 16.8 | 21 | P < 0.01 |
| Theophylline | 1 mM | 496.3 | 630 | P < 0.01 |
| Isoproterenol | 1 μM | 472.3 | 600 | P < 0.01 |

In this test, the basal lipolysis was high (79 μM NEFAs released in 2 hours under these conditions). Theophylline at 1 mM and isoproterenol at 1 μM significantly stimulated lipolysis (630% and 600%, respectively, relative to the control), which allowed the test to be validated. The rice protein hydrolysate significantly reduced the basal lipolysis, by 21% and 41%, respectively, relative to the control, without any apparent inteference of the product with the NEFA assay.

These results thus show that this active agent reduces the lipolytic activity of adipocytes.

### Example 2

### Demonstration of the Properties of Another Rice Protein Hydrolysate on Lipolysis

We evaluated the effect of another rice protein hydrolysate (Colhibin® sold by the company Pentapharm at 14.5% active material) on the hydrolysis of adipocyte triglycerides by measuring the release of non-esterified fatty acids (NEFA) into the medium.

This rice protein hydrolysate comprises peptides with a mean molecular mass ranging from 1000 to 5000 daltons.

We followed the same protocol as that described in Example 1 with an identical test medium, in which were tested, respectively:
the rice protein hydrolysate Colhibin® at a final concentration of 10%, 2% and 0.4%, corresponding, respectively, to 1.45%, 0.29% and 0.058% active material;
theophylline at a final concentration of 1 mM and isoproterenol at a final concentration of 1 μM.

We obtained the following results:

| | Concentration | | | |
|---|---|---|---|---|
| | in the final | NEFA | % activity | |
| Treatment | medium | (μM) | vs Control | p |
| Untreated | — | 33.2 | 100 | — |
| control | | | | |
| Rice protein | 10% | 4.0 | 12 | p < 0.01 |
| hydrolysate | (1.45% of AM) | | | |
| | 2% | 5.0 | 15 | p < 0.01 |
| | (0.29% of AM) | | | |
| | 0.4% | 13.0 | 39 | p < 0.05 |
| | (0.058% of AM) | | | |
| Theophylline | 1 mM | 626.3 | 1888 | p < 0.05 |
| Isoproterenol | 1 μM | 613.3 | 1849 | p < 0.05 |

AM = active material

In this test, the basal lipolysis corresponded to a release of 33.2 μM of non-esterified fatty acids (NEFA) in 2 hours under the untreated control condition.

Theophylline at 1 mM and isoproterenol at 1 μM significantly stimulated lipolysis (by 1888% and 1849%, respectively, relative to the control), which allowed the test to be validated.

The rice protein hydrolysate Colhibin® significantly reduced the basal lipolysis at the 3 concentrations tested, by 12%, 15% and 39% relative to the control, without any apparent interference of the product with the NEFA assay.

These results thus show that this rice protein hydrolysate significantly reduces the lipolytic activity of adipocytes.

The results of Examples 1 and 2 thus show that rice protein hydrolysates comprising peptides of low molecular mass (less than 1400 daltons) or with a molecular weight ranging from 1000 to 5000 daltons significantly reduce the lipolytic activity of adipocytes.

### Example 3

### Anti-Ageing Serum

An O/W emulsion having the composition below is prepared, in a manner that is standard to those skilled in the art:

| | |
|---|---|
| Rice protein hydrolysate in water* | 0.5% |
| Extract of Laminaria ochroleuca in a | 0.1% |
| water/propylene glycol mixture** | |
| Oils | 5% |
| Surfactants | 1% |
| Tensioning agents | 7% |
| Thickeners/gelling agents | 1% |
| Glycerol | 1% |
| Propylene glycol | 1% |
| Alcohol | 13% |
| Nacres | 0.7% |
| Citric acid | 0.1% |
| Preserving agents | qs |
| Water | qs 100% |

*at 6.25% active material, prepared as described in patent application WO 02/102 347
**at 16.3% solids (Laminaine from Secma)

This composition may be applied morning and/or evening to the face to remodel the skin and redefine the contours of the face.

### Example 4

### Care for the Contour of the Eyes

An O/W emulsion having the composition below is prepared, in a manner that is standard to those skilled in the art:

| | |
|---|---|
| Rice protein hydrolysate (Colhibin ® | 0.075% |
| sold by Pentapharm) | |
| C-β-D-xylopyranoside-2-hydroxypropane | 2.5% |
| Oils | 8% |
| Fatty substances | 4% |
| Surfactants | 2% |
| Thickeners/gelling agents | 1.5% |
| Glycerol | 4% |
| Neutralizers | 0.2% |
| Preserving agents | 1.2% |
| Fillers | 3.5% |
| Hexyldecanol | 1.5% |
| Water | qs 100% |

This composition may be applied morning and/or evening to the contour of the eyes to limit the age-related hollowing.

The above written description of the invention provides a manner and process of making and using it such that any person skilled in this art is enabled to make and use the same, this enablement being provided in particular for the subject matter of the appended claims, which make up a part of the original description and including a process for preventing and/or reducing the signs of ageing of the skin, by applying to facial skin a composition containing, in a physiologically acceptable medium, at least one rice protein hydrolysate and at least one agent for increasing glycosaminoglycan synthesis.

As used above, the phrases “selected from the group consisting of,” “chosen from,” and the like include mixtures of the specified materials.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out. Terms such as “contain(s)” and the like as used herein are open terms meaning ‘including at least’ unless otherwise specifically noted.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A process for delaying the onset of the appearance of, and/or for reducing signs of, ageing of the skin, comprising:
applying to the skin of a subject in need thereof a composition comprising, in a physiologically acceptable medium, at least one rice protein hydrolysate and at least one agent for increasing glycosaminoglycan synthesis in an amount effective to reduce signs of ageing.

2. The process according to claim 1, wherein said subject is in need of a remodelling the face and/or a limiting of the age-related hollowing of the face.

3. The process according to claim 1, wherein said subject is in need of redefining the contours of the face and/or limiting the hollowing of the cheeks and/or of the contour of the eyes.

4. The process according to claim 1, wherein peptide(s) contained in the rice protein hydrolysate have a molecular weight of less than or equal to 5000 daltons.

5. The process according to claim 1, wherein more than 50% of the peptides contained in the rice protein hydrolysate have a molecular weight of less than 1400 daltons.

6. The process according to claim 1, wherein at least 40% of the peptides contained in the rice protein hydrolysate with a molecular weight of less than 1400 daltons have a molecular weight of between 75 and 175 daltons.

7. The process according to claim 1, wherein the peptides contained in the rice protein hydrolysate have a mean molecular weight of between 300 and 400 daltons.

8. The process according to claim 1, wherein said rice protein hydrolysate is obtained according to a process comprising the steps of:
dissolving rice proteins in water to obtain a suspension,
hydrolysing this suspension in the presence of one or more enzymes chosen from: bacterial aminopeptidases in neutral or acidic medium, stomach proteases of animal origin in acidic medium, bacterial proteases in neutral medium, bacterial proteases in alkaline medium, extracted from Bacillus licheniformis or Bacillus subtilis, bacterial endoproteases, and pancreatic enzymes of animal origin;
optionally, concentrating the peptides with a molecular mass of less than 5000 daltons in the active fraction to provide a concentrate; and
optionally, performing a sterilizing filtration on the concentrate.

9. The process according to claim 1, wherein the rice protein hydrolysate is derived from the species Oryza sativa.

10. The process according to claim 8, wherein the process for preparing the said rice protein hydrolysate does not comprise a fermentation or germination step.

11. The process according to claim 1, wherein the said rice protein hydrolysate is obtained from a rice fraction free of rice bran.

12. The process according to claim 1, wherein the rice protein hydrolysate represents from 0.01% to 2% by weight relative to the total weight of the composition.

13. The process according to claim 1, wherein the rice protein hydrolysate represents from 0.02% to 0.4% by weight relative to the total weight of the composition.

14. The process according to claim 1, wherein the agent for increasing glycosaminoglycan synthesis is chosen from: D-xylose, its esters and oligosaccharides containing D-xylose; C-glycosides; a product of fermentation of milk with Lactobacillus vulgaris; an extract of brown alga Padina pavonica; an extract of Saccharomyces cerevisiae; an extract of Laminaria ochroleuca; an extract of Centella asiatica; an extract of cress (Nasturtium officinale); and mixtures thereof.

15. The process according to claim 1, comprising an extract of Laminaria ochroleuca.

16. The process according to claim 1, comprising at least one of C-β-D-xylopyranoside-2-hydroxypropane, and the salts and optical and geometrical isomers thereof.

17. The process according to claim 15, wherein the extract of Laminaria ochroleuca is obtained according to a process comprising:
harvesting fresh algae,
washing the algae,
milling the algae, optionally to a size of between 100 and 200 μm,
removing cell debris by centrifugation, to obtain a first supernatant,
fractionating the first supernatant by precipitation with acid, and by adsorption onto activated charcoal, to obtain a second supernatant,
filtering the second supernatant until the filtrate has reached a glycine-betaine concentration, measured by HPLC, of 12.5%±2.5%.

18. The process according to claim 1, wherein the agent for increasing glycosaminoglycan synthesis represents from 0.001% to 5% by weight relative to the total weight of the composition.

19. The process according to claim 15, wherein the extract of Laminaria ochroleuca represents from 0.005% to 0.1% relative to the total weight of the composition.

20. The process according to claim 1, wherein at least a portion of the rice protein hydrolysate and the agent for increasing glycosaminoglycan synthesis are present in niosomes.

21. The process according to claim 1, wherein the composition is in the form of an oil-in-water emulsion.

22. A composition comprising, in a physiologically acceptable medium, at least one rice protein hydrolysate and at least one extract of Laminaria ochroleuca.

23. A composition comprising, in a physiologically acceptable medium, at least one rice protein hydrolysate and at least one agent chosen from C-β-D-xylopyranoside-2-hydroxypropane, and the salts and optical and geometrical isomers thereof.
